(19) United States
(12) Patent Application Publication
(10) Pub. No.: US 2008/0288007 A1
(43) Pub. Date: Nov. 20, 2008

(54) Hygienic-Therapeutic Multiplex Devices

(75) Inventor: Henryk Malak, Ellicott City, MD (US)

Correspondence Address: NIXON & VANDERHYE, PC, 901 NORTH GLEBE ROAD, 11TH FLOOR, ARLINGTON, VA, 22203, US

(73) Assignee: UNITED LABORATORIES & MANUFACTURING, LLC, Sterling, VA (US)

(21) Appl. No.: 12/091,860

(22) PCT Filed: Jun. 15, 2006
(86) PCT No.: PCT/US06/23256

§ 371 Date: Apr. 28, 2008

Related U.S. Application Data
(60) Provisional application No. 60/730,820, filed on Oct. 28, 2005.

(51) Int. Cl. A61N 1/00; A61N 2/00

(52) U.S. Cl. 607/2; 607/96; 607/115

(57) ABSTRACT

Multiplex hygienic-therapeutic devices are based upon plurality energy members arranged in at least one layered structure. The devices comprise energy members including, for example, electromagnetic, sonic, microwave, magnetic, thermal, electric, electrostatic, mechanical, ionic, and ionizing radiation. The use of multiplex hygienic-therapeutic devices with the hygienic and therapeutical substances whose hygienic-therapeutical properties may be enhanced by radiation of the energy member and/or the energy members of the multiplex hygienic-therapeutic devices is also disclosed.

### CROSS REFERENCE TO RELATED PATENT APPLICATION

This application claims the benefit of U.S. Provisional Application Ser. No. 60/730,820 filed Oct. 28, 2005, entitled “Hygienic-Therapeutic Multiplex Devices,” which is incorporated by reference.

### FIELD OF THE INVENTION

This invention is generally related to hygienic, therapeutic, and medical care methods, devices, and applications.

### BACKGROUND OF THE INVENTION

Many consumers perceive a need for hygienic, therapeutic and personal care devices with new features that may enhance hygienic or therapeutic treatments or provide novel hygienic or therapeutic effects.

With respect to oral care, for example, it is generally advisable for a person to brush his teeth every day. Oral care generally has numerous goals, including, for example: removing or eliminating bacterial; removing plaque; whitening teeth; sterilizing the mouth; reducing or eliminating gum bleeding; promoting healthier gums and oral tissue, killing pre-cancerous and cancerous cells; healing wounds; and regenerating tooth and gum nerves; and so forth. Currently available oral care devices—such as toothbrushes, dental toothpastes, and mouthwashes—may not achieve all or even some of these goals. Accordingly, there may be a need for implementing several new features into toothbrushes as well as a need for using new hygienic and therapeutic methods and devices to improve oral care.

Similar needs may exist for other hygienic, therapeutic and personal care devices, including, for example, hairbrushes, combs, hair dryers, and other devices. When adding new features to a device and using new hygienic-therapeutic methods, it is not necessarily the case that the benefits from added features could be estimated additively. The combined features may synergistically create new hygienic-therapeutic modalities in these devices. That is, the efficacy of the combined features may not be effectively predicted by merely summing the efficacy of the individual modalities.

For example, a toothbrush with light and ultrasound features may provide enhanced killing of cancerous cells or bacteria. Indeed, it is believed that applying energy of light and ultrasound to cells, either simultaneously or sequentially may cause an increased up-take of photodynamic substances into the cells or bacteria.

### SUMMARY OF THE INVENTION

This disclosure generally relates to multiplex hygienic-therapeutic devices, such as, for example, oral care devices, hair care devices, and skin care devices, that are built based upon a plurality of energy members arranged in layered structures. The devices include at least two energy members selected from the group consisting of: electromagnetic, sonic, microwave, magnetic, thermal, electric, electrostatic, mechanical, ionic, and ionizing radiation. The layered structures can provide pre-designed radiation distributions of the energy members on/in an irradiated body for hygienic-therapeutic treatment effects and diagnostic capabilities.

The configuration and selection of the members depend upon the type of the device and properties of the treated body. For example, a pain-relief device that is usually in close proximity or direct contact with the body can be assembled with the electromagnetic energy members (light emitting diodes (LEDs) or laser diodes) arranged in a spectral multi-layer structure and with additional sonic wave sources placed within each layer. The spectral multi-layer structure may prevent overheating or burning of the body exposed to this radiation. Such problems may not occur in a different version of the device such as a toothbrush. In a toothbrush, the members can be arranged, for example, in a two-layer structure: one layer with arrayed LEDs/laser diodes and another layer with arrayed sonic sources.

Other arrangements of the members in these devices are possible. Accordingly, the scope of the invention is not limited to these arrangements, and, furthermore, the scope of the invention is not limited to these two devices. The invention includes, for example, pain-relief devices, hygienic-therapeutic illuminators, toothbrushes, proximal toothbrushes, hair removal devices, combs, hairbrushes, hair dryers, dental inserts, ear inserts, bandages, pads, and other hygienic-therapeutic or personal care devices for the skin, mouth, hair, and body.

Also included in the scope of the invention are the devices designed for use with hygienic-therapeutic substances whose hygienic-therapeutical properties may be enhanced by an energy member or members. Such substances may, for example, enhance teeth whitening, remove cancerous and pre-cancerous cells, kill bacteria/viruses, bioactivate tissue, heal wounds, regenerate nerves, without limitation. Furthermore, these substances may be applied externally or internally to the body. For example, such substances used in oral care include, but are not limited to: toothpastes and mouthwashes containing peroxides and/or photodynamic therapy substances.

In accordance with one embodiment, there is a hygienic-therapeutic device comprising: a first set of at least one energy member arranged in a layered structure, wherein the first set of at least one energy member is selected from the group consisting of electromagnetic sonic, microwave, magnetic, thermal, electric, electrostatic, mechanical, ionic, and ionizing radiation; and a second set of at least one energy member arranged in a layered structure, wherein the second set of at least one energy member is selected from the group consisting of electromagnetic sonic, microwave, magnetic, thermal, electric, electrostatic, mechanical, ionic, and ionizing radiation; wherein the first set of at least one energy member contains only a single type of energy member; wherein the second set of at least one energy member contains only a single type of energy member; and wherein the first set of at least one energy member does not contain the same type of energy member as the second set of at least one energy member.

In accordance with another embodiment, the hygienic-therapeutic device is a pain-relief device, a toothbrush, a hygienic-therapeutic illuminator, a hair removal device, a comb, a hairbrush, a hair dryer, a dental insert, an ear insert, a bandage, or a pad. Preferably, the hygienic-therapeutic device is a toothbrush. More preferably, the toothbrush comprises a first set of at least one energy member consisting of at least one electromagnetic energy source, and a second set of at least one energy member consisting of at least one sonic wave source.

### BRIEF DESCRIPTION OF THE DRAWINGS

The accompanying drawings, which are incorporated in, and constitute a part of the specification, illustrate or exemplify embodiments of the present invention and, together with the description, generally explain the principles and features of the present invention.

FIG. 1 illustrates a hand-held pain-relief device in accordance with an embodiment of the present invention.

FIG. 2 illustrates a toothbrush in accordance with an embodiment of the present invention.

FIG. 3 illustrates a cosmetic/phototherapy illuminator in accordance with another embodiment of the present invention.

FIG. 4 illustrates a proximal toothbrush in accordance with another embodiment of the present invention.

FIG. 5 illustrates a hair removal device in accordance with another embodiment of the present invention.

FIG. 6 illustrates a comb/hair brush in accordance with another embodiment of the present invention.

FIG. 7 illustrates a hair dryer in accordance with another embodiment of the present invention.

FIG. 8 illustrates a fragment of a dental insert in accordance with another embodiment of the present invention.

FIG. 9 illustrates an ear insert of a bandage in accordance with another embodiment of the present invention.

FIG. 10 illustrates a fragment of a bandage in accordance with another embodiment of the present invention.

FIG. 11 illustrates a pad in accordance with another embodiment of the present invention.

FIG. 12 illustrates a toothbrush in accordance with another embodiment of the present invention.

FIG. 13 illustrates a hair removal/shaving device in accordance with another embodiment of the present invention.

FIG. 14 illustrates a hand-held pain-relief device in accordance with another embodiment of the present invention.

FIG. 15 illustrates a toothbrush in accordance with yet another embodiment of the present invention.

FIG. 16 illustrates a toothbrush in accordance with yet another embodiment of the present invention.

FIG. 17 illustrates a toothbrush in accordance with yet another embodiment of the present invention.

FIGS. 18a and 18b illustrate a toothbrush in accordance with yet another embodiment of the present invention.

### DESCRIPTION OF PREFERRED EMBODIMENTS

All patents, patent applications, and literatures cited or referenced in this description are incorporated herein by reference in their entireties. In the case of inconsistencies, the present disclosure, including definitions and usage, will control.

This disclosure generally describes novel multiplex hygienic-therapeutic or personal care devices that are based upon energy members arranged in a layered structure. As denoted in the figures, embodiments of the present invention consist of at least one energy member selected from the group consisting of: electromagnetic 101, sonic 102, microwave 103, magnetic 104, thermal 105, electric 106, electrostatic 107, mechanical 108, ionic 109, and ionizing radiation 100. The layered structures may provide designed/controlled radiation distributions of plurality energy members on/in a treated body for unique hygienic-therapeutic treatment effects and diagnostic capabilities. Each layer of the multi-layer structure in the device can be independently controlled by electronics 111 and computer or processor 112 and according to hygienic-therapeutic needs. The energy members in the devices may work in pulse/frequency modes with variable pulse durations and variable frequencies or in a CW (continuous wave) mode, or in a combination of pulse/frequency and CW modes.

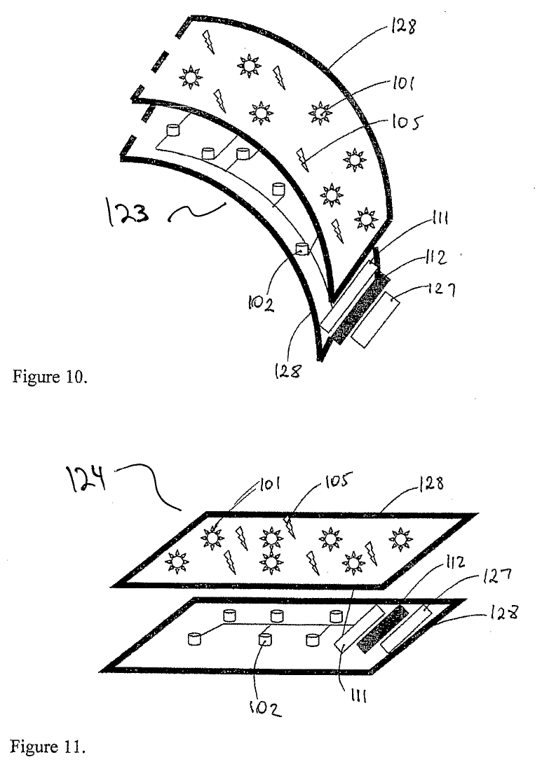

It should be understood that the figures illustrate certain components (i.e., electronics 111, a computer or processor 112, and a power supply 127) as contained within housing 128. In alternative embodiments, however, these components may be partially or wholly external to the particular devices. For example, electronics 111, a computer or processor 112, and a power supply 127 may be partially or wholly external to the housing 128 of the bandage 123 as illustrated in FIG. 10. It should also be noted that the housing may comprise a unitary structure or multiple structures.

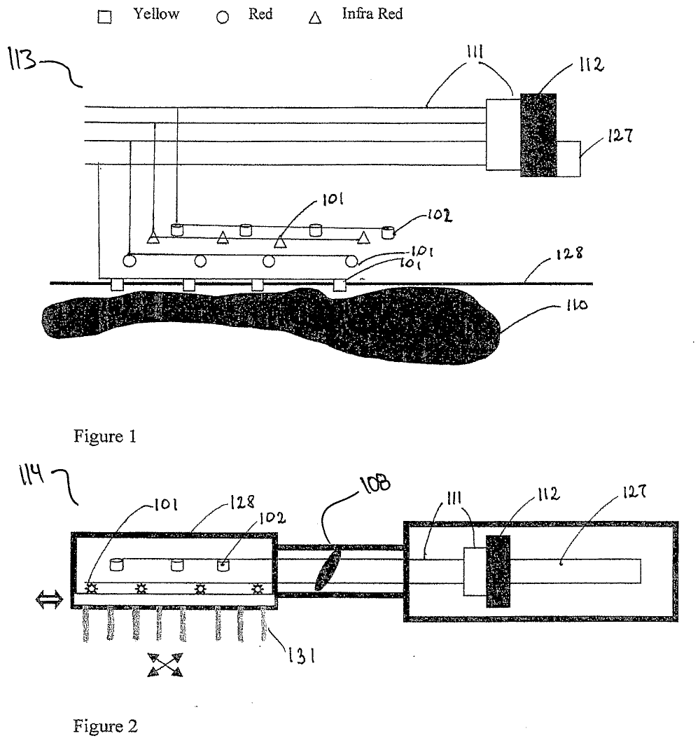

The scope of the invention includes, for example, multiplex hygienic-therapeutic/personal care devices selected from the group consisting of: pain-relief device 113, toothbrush 114, hygienic-therapeutic illuminator 115, proximal toothbrush 116, hair removal device 117, comb 118, hairbrush 119, hair dryer 120, dental insert 121, ear insert 122, bandage 123, pad 124, and other hygienic-therapeutic/personal care devices. Examples of the devices with the different arrangements and selections of the energy members in these devices are illustrated in FIGS. 1-17.

In an embodiment, the invention considers the use of the electromagnetic energy member as a single electromagnetic source or a plurality of electromagnetic sources selected from the group consisting of: light emitting diodes, super luminescence diode, organic luminescence diode, laser diode, vertical cavity laser, laser, black body radiation, luminescence, fluorescence, phosphorescence, bioluminescence, chemiluminescence, electroluminescence, X-Ray, ionizing radiation, and microwave.

The selection and arrangement of the members in the layered structure and their technical parameters depend upon the type of hygienic-therapeutic device. For example, as depicted in FIG. 1, a pain-relief device 113, which may be in close proximity or in direct contact with the body, has light emitting diodes (LEDs) and/or laser diodes 101 preferably arranged in a spectral layered structure with additional sonic wave sources 102 placed in one of the layers. The spectral layered structure may prevent overheating or burns of the exposed body to electromagnetic radiation. Each wavelength of electromagnetic radiation has different heat energy content. Accordingly, sources with NIR and IR wavelengths should generally be placed at a further distances from the body than should blue, green, and/or orange wavelengths sources. Such problems may not occur in a different type of the device, such as a toothbrush. As illustrated, this embodiment of pain-relief device 113 also contains electronics 111, a computer or processor 112, and a power supply 127. These components are contained within housing 128 of the pain-relief device. The electronics 111 and processor 112 control the electromagnetic energy members 101 and sonic wave sources 102. Electromagnetic energy members 101 and sonic wave sources 102 can be directed to impact human 110.

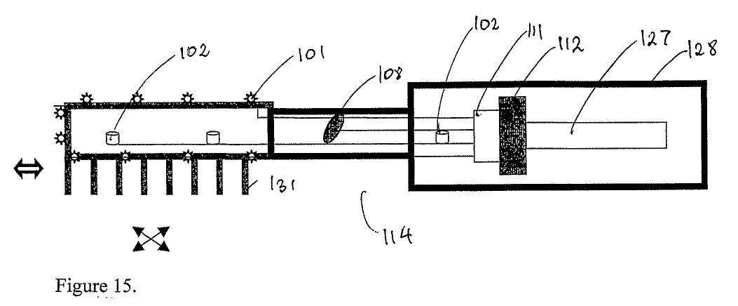

FIG. 2 illustrates an embodiment related to toothbrush 114. In this case, the members can be arranged only in a two-layer structure; one layer with arrayed LEDs/laser diodes 101 and another layer with arrayed sonic sources 102. A bristle head 131 is designed to minimally disturb radiation of the light and sonic sources and can be optically transparent (such as depicted in FIG. 15) and replaced at the end of the bristles lifespan. As illustrated, this embodiment of a proximal toothbrush also contains electronics 111, a computer or processor 112, a mechanical energy source 108, and a power supply 127. The mechanical energy source 108 causes motion, such as vibration, back-and-forth and/or up-and-down, to assist the proximal toothbrush clean the oral cavity.

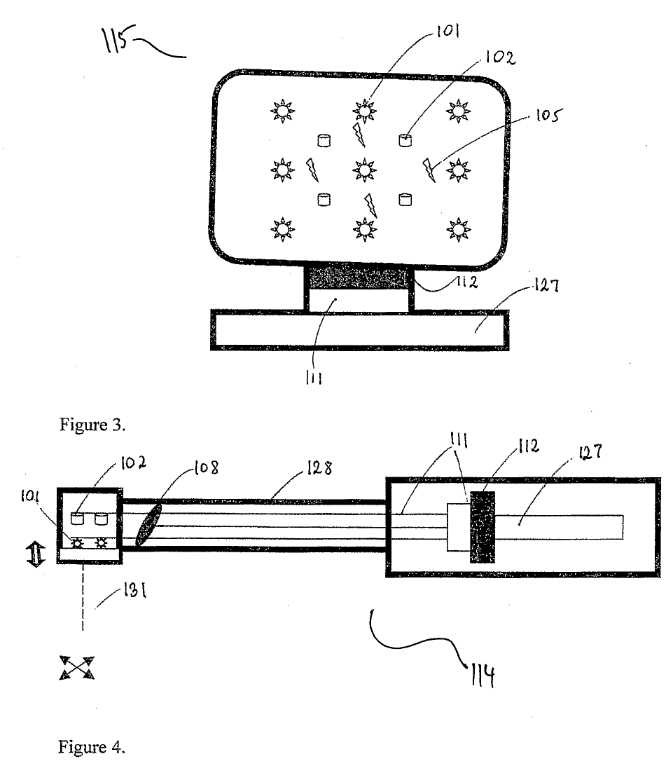

FIG. 3 illustrates a cosmetic/phototherapy illuminator 115 in which a multiplex energy source has arranged LEDs/laser diodes 101, sonic wave sources 102, and infrared energy (thermal) sources 105 in a multi-layer structure. As illustrated, this embodiment also contains electronics 111, a computer or processor 112, and a power supply 127. These components are contained within housing 128 of the cosmetic/phototherapy illuminator 115.

FIG. 4 illustrates a proximal toothbrush 114 in which a multiplex energy source has arranged energy sources in a two-layer structure: one layer with an array of LEDs/laser diodes 101 and another layer with an array of sonic wave sources 102. As illustrated, this embodiment of a proximal toothbrush also contains electronics 111, a computer or processor 112, a mechanical energy source 108, and a power supply 127. The mechanical energy source 108 causes motion, such as vibration, back-and-forth and/or up-and-down, to assist the proximal toothbrush clean the oral cavity. These components are contained within housing 128 of the toothbrush 114.

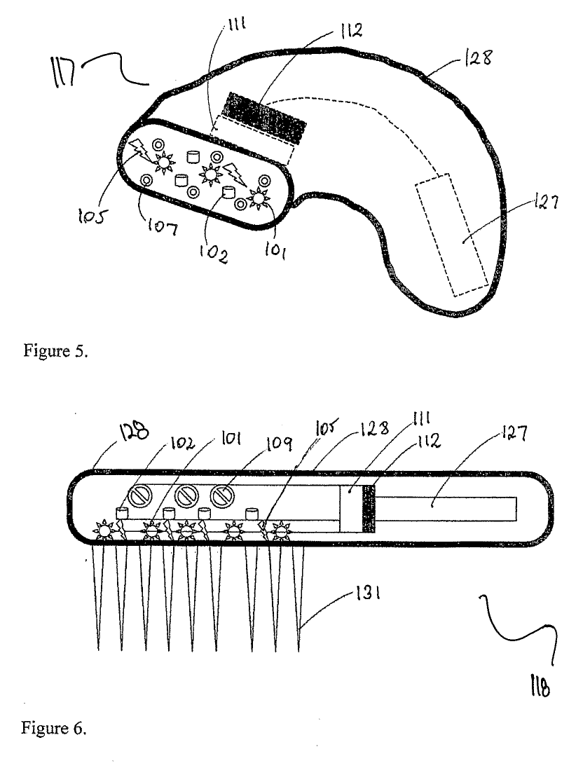

FIG. 5 illustrates a hair removal device 117 in which a multiplex energy source has arranged energy sources in a two-layer structure: one layer with an array of electrostatic sources 107 and thermal sources 105, and a second layer with an array of laser diodes 101 and sonic wave sources 102. As illustrated, this embodiment of a hair removal device also contains electronics 111, a computer or processor 112, and a power supply 127. These components are contained within housing 128 of the hair removal device 117. Hair removal device 117 may be, for example, a razor.

FIG. 6 illustrates a comb/hair brush 118 in which a multiplex energy source has arranged energy sources in a three-layer structure: one layer with an array of LEDs/laser diodes 101 and far-infrared (thermal) sources 105, another layer with an array of sonic wave sources 102, and another layer with an array of ionic sources 109. As illustrated, this embodiment of a comb/hair brush also contains electronics 111, a computer or processor 112, and a power supply 127. These components are contained within housing 128 of the comb/hair brush 118. Bristles 131 are attached to housing 128.

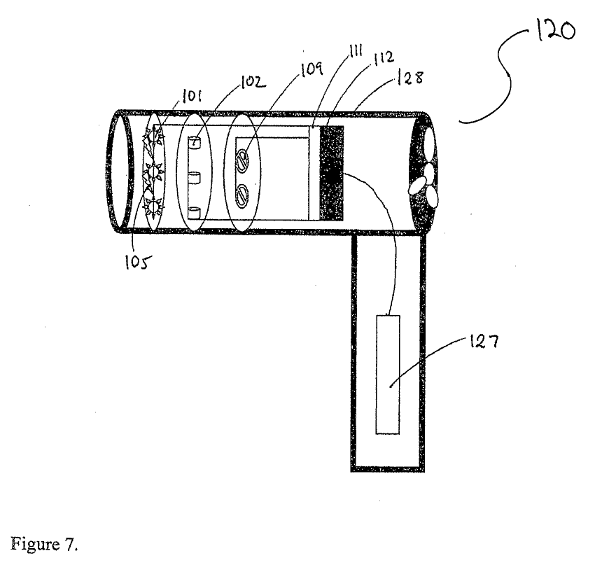

FIG. 7 illustrates a hair dryer 120 in which a multiplex energy source has arranged energy sources in a three-layer structure: one layer with an array of LEDs/laser diodes 101 and far-infrared (thermal) sources 105, another layer with an array of sonic sources 102, and another layer with an array of ionic sources 109. As illustrated, this embodiment of a hair dryer also contains electronics 111, a computer or processor 112, and a power supply 127. These components are contained within housing 128 of the hair removal device 120.

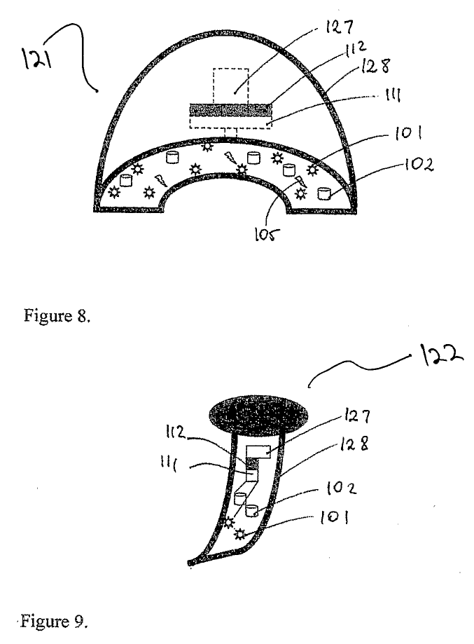

FIG. 8 illustrates a fragment of a dental insert 121 in which a multiplex energy source has arranged energy sources in a two-layer structure: one layer with an array of LEDs/laser diodes 101 and far-infrared (thermal) sources 105, and another layer with an array of sonic sources 102. As illustrated, this embodiment of a hair dryer also contains electronics 111, a computer or processor 112, and a power supply 127. These components are contained within housing 128 of the dental insert 121.

FIG. 9 illustrates an ear insert 122 in which a multiplex energy source has arranged energy sources in a two-layer structure; one layer with an array of LEDs/laser diodes 101, and another layer with an array of sonic sources 102. As illustrated, this embodiment of a hair dryer also contains electronics 111, a computer or processor 112, and a power supply 127. These components are contained within housing 128 of the ear insert 122.

FIG. 10 illustrates a fragment of a bandage 123 in which a multiplex energy source has arranged energy sources in a two-layer structure: one layer with an array of LEDs/laser diodes 101 and far-infrared (thermal) sources 105, and a second layer with an array of sonic sources 102. As illustrated, this embodiment of a hair dryer also contains electronics 111, a computer or processor 112, and a power supply 127. These components (i.e., electronics 111, a computer or processor 112, and a power supply 127) are illustrated as contained within housing 128. In this and all other embodiments, these components may be external to the particular devices. Thus, electronics 111, a computer or processor 112, and a power supply 127 may be partially or wholly external to the housing 128 of the bandage 123.

FIG. 11 illustrates a pad 124 in which a multiplex energy source has arranged energy sources in a two-layer structure: one layer with an array of LEDs/laser diodes 101 and far-infrared (thermal) sources 105, and another layer with an array of sonic sources 105. As illustrated, this embodiment of a hair dryer also contains electronics 111, a computer or processor 112, and a power supply 127. These components are illustrated as contained within housing 128.

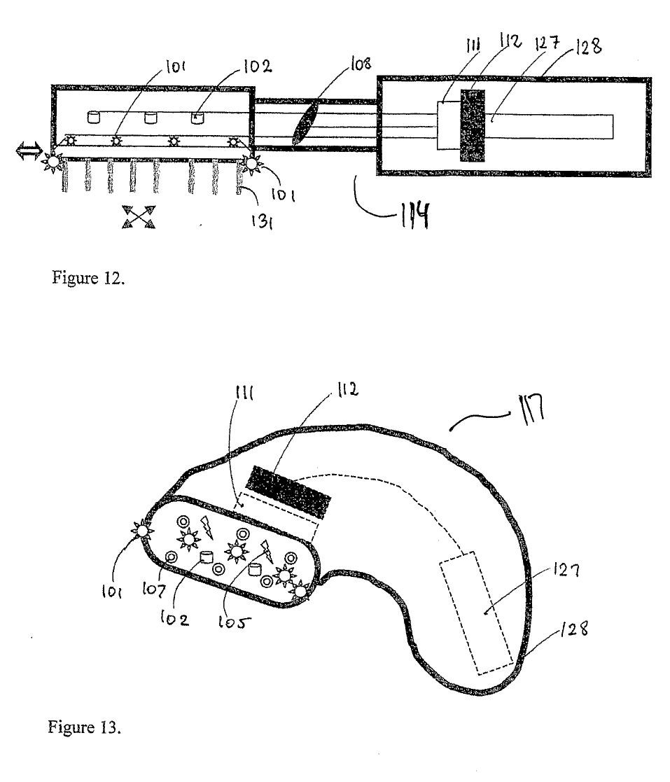

FIG. 12 illustrates a toothbrush 114 in which an electromagnetic source 101, such as, for example, UV LED, is used to sterilize bristle head 131. This device is similar to the device illustrated in FIG. 2 In this embodiment, electromagnetic source 101 may be embedded on or external to the surface of housing 128.

FIG. 13 illustrates a hair removal device 117 in which an electromagnetic source 101, for example, UV LED, is used in to sterilize bacteria and razor head. This device is similar to the hair removal device 117 illustrated in FIG. 5. In this embodiment (FIG. 13), electromagnetic source 101 may be embedded on or external to the surface of housing 128.

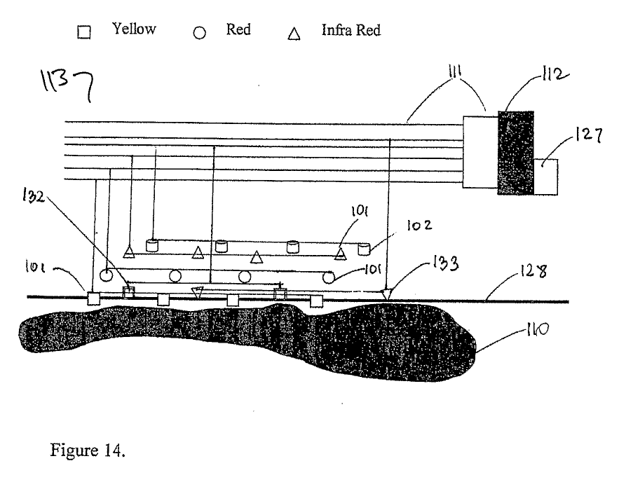

FIG. 14 illustrates a hand-held pain-relief device 113 with a diagnostic feedback in which a multiplex energy source has arranged LEDs/laser diodes 101 in a spectral multi-layer structure with additional sonic sources 102, photon detectors 132 and thermocouples 133 placed in one of the layers. This device is similar to the pain-relief device illustrated in FIG. 1.

FIG. 15 illustrates a toothbrush 114 with an optically transparent bristle head, in which a multiplex energy source has arranged energy sources in a three-layer structure: one layer with an array of LEDs/laser diodes 101, second layer with an array of sonic sources 102, and a third layer with an array of LEDs/laser diodes 101. As illustrated, a sonic source 102 is placed in a toothbrush handle. This device is similar to the toothbrush 114 illustrated in FIG. 2.

As illustrated in FIG. 15, light and ultrasound radiate in multiple directions, which may allow performance of a hygienic function and photodynamic therapy to entire oral cavity. The bristles can also deliver sonic waves to the body; therefore, the sonic source also can be placed in a toothbrush handle and sonic waves may propagate to the bristle head through a toothbrush housing 128. Other arrangements and selections of the energy members in these devices are also possible, and the scope of the invention is not limited to these arrangements.

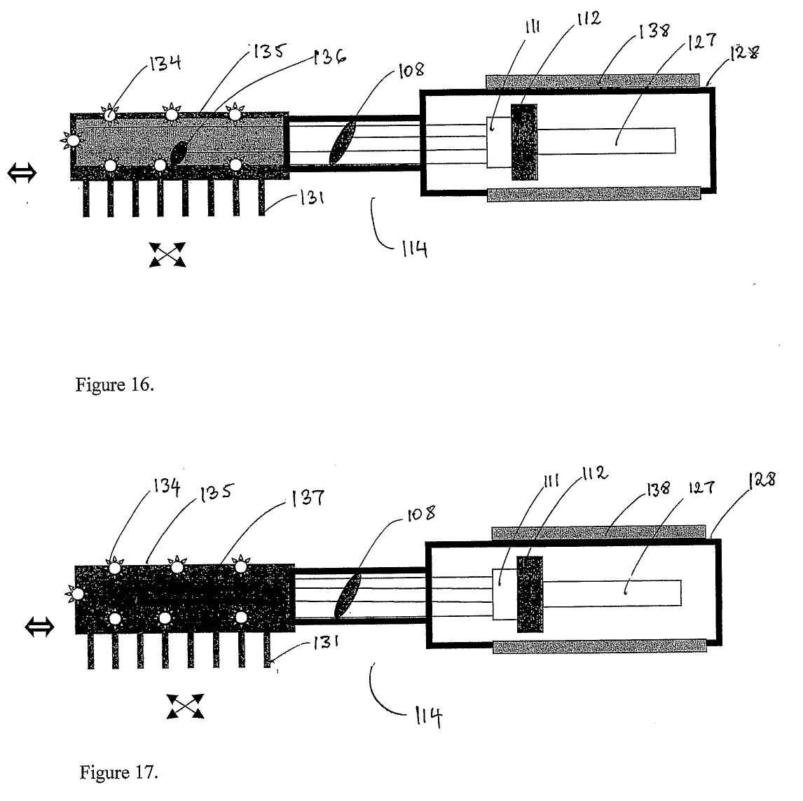

FIG. 16 illustrates a toothbrush 114 with energy sources arranged in a three-layer structure: one layer with an array of LEDs/laser diodes 134 emitting light at ultrasonic frequencies through the bristlehead, a second layer with a small motor 136 providing micro-vibrations, and a third layer with an array of LEDs/laser diodes 134 emitting light at ultrasonic frequencies. The toothbrush has also antibacterial surfaces 135 and a grip improving material 138 placed on the toothbrush handle. This device is similar to the toothbrush 114 illustrated in FIG. 2.

FIG. 17 illustrates a toothbrush with energy sources arranged in a three-layer structure: one layer with an array of LEDs/laser diodes 134 emitting light at ultrasonic frequencies through the bristle head, second layer with a wire resonator 137 providing micro-vibrations, and a third layer with an array of LEDs/laser diodes 134 emitting light at ultrasonic frequencies. The toothbrush has also antibacterial surfaces 135 and a grip improving material 138 placed on the toothbrush handle. This device is similar to the toothbrush 114 illustrated in FIG. 2.

As illustrated in FIGS. 16 and 17, the energy members—such as light or electromagnetic, ultrasonic, or thermal—may be generated by the same type of source—such as LEDs or laser diodes. In other words, a LED may, for example, be a source for an electromagnetic, sonic, or thermal energy member. Alternatively, a LED may generate a combination of at least two energy members. Accordingly, the energy members may be generated by the same type of source or even same energy source.

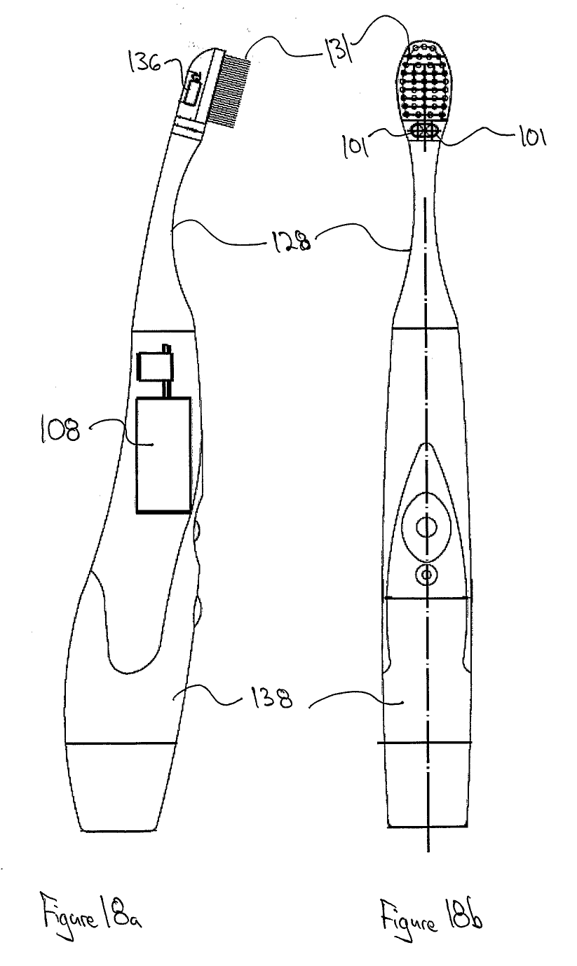

FIGS. 18a and 18b illustrate a toothbrush with multiple energy sources arranged in a layered structure. One layer consists of two LEDs/laser diodes 101 within the bristle head. Another layer consists of small motor 136 disposed within the bristle head. A third energy source is the mechanical energy source 108 located in the body of the toothbrush. The toothbrush also, for instance, has bristles 131, housing 128 and grip material 138. Although not illustrated, the toothbrush may further comprise an antibacterial surface. As illustrated, housing 128 comprises multiple structures.

All the components in toothbrushes are commonly available, off-the-shelf items. The LEDs/laser diodes may be obtained from Nichia. Preferrably, the LEDs are 3 mm LEDs, BUWLCW40BB09CO7, available from Besthongkong Technology Co. Ltd. (www.besthongkong.com). In addition, the LEDs may be 4 MHz sonic signal feeding LED generated by standard electronics. Preferrably, the power source is a 4.8 V Ni-MH JS800AA rechargeable battery, made by Jianshun (www.jianshun.com). The sonic transducers may be obtained from Toshiba. The small vibration motors may be obtained from Jinlong Machinery or from an electronics store. Preferrably, the vibration motor may be model SF made by Sunzheng Group Co., Ltd. (www.sunzheng.com). Antibacterial substances may be obtained from PChem Associates, Inc. Thermoplastic materials may be obtained from Advanced Elastomer Systems, LP (or Santoprene).

Although the invention has been explained in relation to the above embodiments having multi-layer configurations of the members in the multiplex energy sources in the devices, a single-layer structure or a double-layer structure is contemplated as within the scope of the invention. A single-layer structure, for example, may contain two or more sets of energy members that may have different penetration depths due to an adjustment to the energy members' technical parameters. Indeed, the parameters of the individual energy members may be adjusted individually and independently, regardless of the precise layered configuration.

Furthermore, devices within the scope of the present invention can be provided with an additional feature or features that can be essential for their effective performance and ease of use. Such features can be: a computer or processor to run electronics and programs for hygienic, therapy, and diagnostic needs; biomedical and instrumental feedbacks with biomedical diagnostic sensors controlled by the computer or processor and associated electronics; a battery or AC/DC power supply; a housing with transparent or semitransparent properties to some members of the multiplex energy source; a data/control local and remote communication medium; a visible light source embedded to the device providing illumination of the body during the use of the device; and other features known in the art.

The scope of the invention also includes the use of multiplex hygienic-therapeutic devices with the hygienic and therapeutical substances whose hygienic-therapeutical properties may be enhanced by radiation of the energy member and/or the energy members of the multiplex hygienic-therapeutic devices. Such substances may enhance teeth whitening, remove cancerous and pre-cancerous cells, kill bacteria/viruses, bioactivate of tissue, heal wounds, and regenerate nerves, but are not limited to them. These substances can be applied externally or internally to the body. Examples of such substances used in oral care are toothpastes and mouthwashes containing peroxides or photodynamic therapy substances, but are not limited to them.

In another embodiment, a device is provided in which some members of the device are used to maintain proper conditions of the device and to enhance performance of the device, and/or for more friendly use. For example, as is shown in FIGS. 12 and 13, a toothbrush and a hair removal/shaving device use an electromagnetic source (UV LED) 101 to sterilize bacteria/viruses on these devices. The sterilization process may be activated manually or by embedded software. The hair removal device or the other hygienic-therapeutic/personal care devices can also be further provided with a visible LED source 130 for illumination of the body during the use of the device in low light conditions. Some members of the multiplex energy source can also warm up the device to body temperature or above to enhance hygienic-therapeutic effects. For example, the hair removal/shaving device 117 can be further provided with a heated blade combined with ultrasonic vibrations which may produce a clean and easy cut through wet or dry hair, avoiding split ends, and frizziness.

A person of ordinary skill in the art will appreciate the optional use of the multiplex hygienic-therapeutic device as a biomedical diagnostic device. The device is built based upon the multiplex energy source in which the members are placed in the layered structure to preserve the designed radiation distribution of the members on/in the treated body. Accordingly, biomedical sensors implemented into the layered structure of the device may provide a quantitative biofeedback from the radiated body, which then can be used for biomedical diagnostics. FIG. 14 illustrates an example of the placement of photon detectors 132 and thermocouples 133 in the layered structure of the pain-relief device.

In yet another embodiment a design of the instrument is provided in which the members in the device can be selected according to properties of the body. In hygienic-therapeutic treatments, blond hair or light skin may require the use of different members or the same members but with different technical parameters than dark hair or skin. Therefore, in the invented device each layer with the plurality energy members may be controlled independently, thus allowing for selection of the energy members and technical parameters of the energy members according to hygienic-therapeutic needs and properties of the body.

FIGS. 16 and 17 illustrate toothbrushes 114 in which ultrasound and light is generated by LEDs/lasers 101 and micro-vibrations in the toothbrush bristle are generated by an additional small motor 136 or a resonator 137. The resonator 137 can provide different type vibrations to the bristle head, including, for example, mechanical, electrical or magnetical. The shape of resonator and material of which is made may be designed to promote one type versus other types of the vibrations. As illustrated, the toothbrushes are further provided with antibacterial surfaces 135 and a grip material 138 that is placed on the toothbrush handles to facilitate improved handling.

An operational mode of the members in the device can be coincidental or consecutive or random. The members may work in variable time, and in a CW mode and/or pulse of frequency mode with variable pulse durations and variable frequencies, respectively. Selection of the mode may depend on the properties of the treated body and hygienic-therapeutical needs, and the mode or modes can be executed in the device manually or automatically with pre-programmed options.

While the invention has been described in connection with the most practical embodiments, it is to be understood that such embodiments are merely preferred and the invention is not to be limited to the disclosed embodiments, but on the contrary, is intended to cover various modifications and equivalent arrangements included within the spirit and scope of the appended claims.

1. A hygienic-therapeutic device comprising:
a first set of at least one energy member arranged in a layered structure, wherein the first set of at least one energy member is selected from the group consisting of electromagnetic sonic, microwave, magnetic, thermal, electric, electrostatic, mechanical, ionic, and ionizing radiation; and
a second set of at least one energy member arranged in a layered structure, wherein the second set of at least one energy member is selected from the group consisting of electromagnetic sonic, microwave, magnetic, thermal, electric, electrostatic, mechanical, ionic, and ionizing radiation;
wherein the first set of at least one energy member contains only a single type of energy member;
wherein the second set of at least one energy member contains only a single type of energy member; and
wherein the first set of at least one energy member does not contain the same type of energy member as the second set of at least one energy member.

2. The hygienic-therapeutic device of claim 1, wherein the hygienic-therapeutic device is a pain-relief device, a toothbrush, a hygienic-therapeutic illuminator, a hair removal device, a comb, a hairbrush, a hair dryer, a dental insert, an ear insert, a bandage, or a pad.

3. The hygienic-therapeutic device of claim 1, wherein the first set of at least one energy member and the second set of at least one energy member operate coincidentally or non-coincidentally with respect to each other.

4. The hygienic-therapeutic device of claim 1 further comprising a third set of at least one energy member arranged in a layered structure, wherein the third set of energy members are selected from the group consisting of electromagnetic sonic, microwave, magnetic, thermal, electric, electrostatic, mechanical, ionic, and ionizing radiation;
wherein the third set of at least one energy member contains only a single type of energy member; and
wherein the third set of at least one energy member does not contain the same type of energy member as the first set of at least one energy member.

5. The hygienic-therapeutic device of claim 1, wherein the first set of at least one energy member is an un-coherent energy source.

6. The hygienic-therapeutic device of claim 1, wherein the first set of at least one energy member is a coherent energy source.

7. The hygienic-therapeutic device of claim 1, wherein the first set of at least one energy member has at least one hygienic or therapeutic function.

8. The hygienic-therapeutic device of claim 1, wherein the layered structure of the first set of at least one energy member and the layered structure of the second set of at least one energy member are the same layered structure.

9. The hygienic-therapeutic device of claim 1, wherein the layered structure of the first set of at least one energy member and the layered structure of the second set of at least one energy member are different layered structures.

10. The hygienic-therapeutic device of claim 1, wherein the device is further provided with a hygienic substance or a therapeutic substance.

11. The hygienic-therapeutic device of claim 1, wherein the device is partially or wholly coated with an anti-bacterial substance.

12. The hygienic-therapeutic device of claim 1 further comprising a grip improving material.

13. The hygienic-therapeutic device of claim 1, wherein the device is further provided with a computer or processor to control the device's hygienic or therapeutic effect.

14. The hygienic-therapeutic device of claim 1, wherein the device is further provided with a diagnostic feedback.

15. The hygienic-therapeutic device of claim 2, wherein the hygienic-therapeutic device is a toothbrush.

16. The hygienic-therapeutic device of claim 15, wherein the first set of at least one energy member consists of at least one electromagnetic energy source, and wherein the second set of at least one energy member consists of at least one sonic wave source.

17. The hygienic-therapeutic device of claim 15 comprising a bristle head, wherein the first set of at least one energy member consists of at least one electromagnetic energy source, and wherein the second set of at least one energy member is a motor disposed within the bristle head.

18. The hygienic-therapeutic device of claim 2, wherein the hygienic-therapeutic device is a comb or a hairbrush.

19. The hygienic-therapeutic device of claim 1 further comprising a transparent or semitransparent housing.

20. The hygienic-therapeutic device of claim 4, wherein the third set of at least one energy member is partially or wholly external to a housing.

21. The hygienic-therapeutic device of claim 1, wherein the first set of at least one energy member and the second set of at least one energy member are generated from the same type of energy source.

22. The hygienic-therapeutic device of claim 21, wherein the first set of at least one energy member and the second set of at least one energy member are generated from the same energy source.
